SEQUENCE LISTING (1 sequence)

SEQ ID NO: 1 - protein, 28 aa, Artificial Sequence
  TA1
SDAAVDTSSEITTKDLKEKKEVVEEAEN

(19) United States
(12) Patent Application Publication
(10) Pub. No.: US 2014/0213506 A1
(43) Pub. Date: Jul. 31, 2014

(54) THYMOSIN ALPHA PEPTIDE FOR PREVENTING, REDUCING THE SEVERITY OF, AND TREATING INFECTION

(71) Applicant: Cynthia W. Tuthill, Menlo Park, CA (US)

(72) Inventor: Cynthia W. Tuthill, Menlo Park, CA (US)

(21) Appl. No.: 13/984,681

(22) PCT Filed: Feb. 3, 2012
(86) PCT No.: PCT/US12/23775

§ 371 Date: Nov. 15, 2013

Related U.S. Application Data
(60) Provisional application No. 61/441,250, filed on Feb. 9, 2011.

(51) Int. Cl. A61K 38/22; A61K 45/06

(52) U.S. Cl. CPC A61K 38/2292; A61K 45/06
USPC 514/2.6; 514/2.4; 514/2.8; 514/2.7

(57) ABSTRACT

The present invention provides methods for preventing, treating, or reducing the severity of infection, including bacterial, viral, and fungal infections, and including infections of more complex etiology. The invention involves the administration of an alpha thymosin peptide regimen, so as to prime or enhance a patient's immune response for pathogen exposure. In certain embodiments, the alpha thymosin regimen is scheduled or timed with respect to potential or expected pathogen exposures. The regimen of alpha thymosin peptide as described herein provides the patient with a more robust immune response to pathogen exposure, including higher antibody titers and/or a more rapid antibody response. In certain embodiments, the patient is immunodeficient or immunecompromised, and/or the patient is hospitalized or scheduled for hospitalization, such that the regimen of alpha thymosin peptide helps to protect the patient from, or reduce the severity of nosocomial infection or illness.

### PRIORITY

ThIS application claims the priority and benefit of U.S. Provisional Application No. 61/441,250, filed Feb. 9, 2011, which is hereby incorporated by reference in its entirety.

### FIELD OF THE INVENTION

The present invention relates to the field of infection, including prevention of, reduction in severity, or treatment of infection, including acute and hospital-acquired infections, and including for immune-compromised patients such as the elderly and chronically

### BACKGROUND

Hospital-acquired infections, such as pneumonia and sepsis, are responsible for significant patient mortality and morbidity, and add significantly to the overall cost of healthcare [Michael Klompas, Prevention of ventilator-associated pneumonia, Expert Rev. Anti Infect. Ther. 8(7), 791-800 (2010); Wheeler D S at al., Novel Pharmacologic Approaches to the Management of Sepsis: Targeting the Host Inflammatory Responses, Recent Pat. inflamm. Allergy Drug Discov. 3(2):96-112 (2009)]. In fact, sepsis was reported as the 10^{th} leading cause of death in 2004 [Wheeler at al. (2009)]. Hospital-acquired infections are further exacerbated by the ever-increasing prevalence of drug resistant microorganisms, which places continual pressure on the conventional antibiotic arsenal. Preventing and/or treating infection, including hospital-acquired infections, is therefore an ongoing need.

A strong and rapid immune response to pathogens is important for preventing or reducing the severity of many acute infections and illnesses, including acute viral, bacterial, and fungal infections. For example, humoral responses against respiratory syncytial virus (RSV) surface proteins play a large role in preventing RSV infection, which is often hospital-acquired, as well as the resolution of infection [Olson and Varga, Pulmonary immunity and immunopathology: lessons from respiratory syncytial virus, Expert Rev. Vaccines 7(8):1239-1255 (2008)]. In fact, inducing a rapid and strong antibody response to a pathogen challenge is a primary goal of most vaccinations. However, it is not possible or cost effective to vaccinate individuals for all potential pathogens, especially those pathogens that may manifest as nosocomial infections such as pneumonia or sepsis, and especially for immunocompromised patients.

A means for strengthening initial immune responses to pathogens in a convenient and cost effective manner is of great need to reduce the impact of infection, including nosocomial infection, and to reduce the rate, mortality, and morbidity associated with such infections.

### SUMMARY OF THE INVENTION

The present invention provides methods for preventing, treating, or reducing the severity of infection, including bacterial, viral, and fungal infections, and including infections of more complex or unknown etiology. The invention involves the administration of an alpha thymosin peptide regimen, so as to prime or enhance a patient's immune response for pathogen exposure.

In certain embodiments, the alpha thymosin regimen is an efficient regimen, which involves relatively few administrations of the agent, and/or is spaced in time to maximize therapeutic and cost effectiveness, and/or is scheduled or timed with respect to potential pathogen exposures. The regimen of alpha thymosin peptide as described herein provides the patient with a more robust immune response to pathogen exposure, including higher antibody titers and/or a more rapid antibody response, and provides such advantages for up to about 50 days with as few as one or two administrations of alpha thymosin. In certain embodiments, the patient is immunodeficient or immunecompromised, and/or the patient is hospitalized or scheduled for hospitalization, such that the regimen of alpha thymosin peptide helps to protect the patient from, or reduce the severity of, nosocomial infection or illness during the period of hospitalization.

More particularly, in one aspect, the invention provides a method for preventing or reducing the severity of an infection that may result from an anticipated pathogen exposure or opportunistic environment. The method comprises administering an efficient regimen of thymosin alpha peptide (e.g., thymosin alpha 1 or “TA1”) to the patient. Generally, at the time of initiating the alpha thymosin regimen, the patient has not been diagnosed with, or is not showing signs or symptoms of, an infection. In certain embodiments, at the time of initiating the alpha thymosin regimen, the patient is being admitted to a hospital or healthcare facility, and/or is scheduled for surgery or invasive medical procedure, or is in need of an invasive medical device (e.g., a ventilator). In such embodiments, the invention enhances the immune response to this inevitable increase in microbial exposure and/or introduction of an opportunistic environment for certain pathogens, thereby preventing or reducing the severity of the resulting infection.

In another aspect, the invention provides a method for treating an infection by administering an alpha thymosin regimen. In this aspect, the patient has been diagnosed as having an infection, such as an acute respiratory, systemic, urinary, or local infection of the skin or a mucosal surface. The infection may be of bacterial, viral, fungal, or mixed or unknown etiology. The infection may be hospital-acquired, and may manifest as sepsis, pneumonia, urinary tract infection, endocarditis, osteomyelitis, or other condition. In some embodiments, the infection involves a drug resistant microorganism, such as Staphylococcus aureus. Pseudomonas sp., E. coli, Klebsiella sp., and/or Clostridium Difficile. The alpha thymosin regimen may be administered concurrently with the standard of care, such as antibiotic or antiviral therapy. In accordance with this aspect of the invention, the alpha thymosin regimen reduces the duration of the infection, and/or reduces the duration of required antibacterial, antiviral, or antifungal treatment. In certain embodiments, the regimen is given, or continued, or repeated after apparent resolution of the infection, to help prevent recurrence after antibiotic or antiviral therapy is complete.

Whether to prevent or treat an infection, the thymosin peptide (e.g., TA1) is administered to the patient with a regimen that is sufficient to enhance the immune response to pathogen exposure. For example, an efficient regimen of thymosin peptide is administered to a human patient at a dose, frequency, and/or timing with respect to an event predicted to lead to pathogen exposure, so as to protect or treat the patient. The efficient regimen is sufficient to treat or protect the patient for up to 50 days with as few as one or two administrations of alpha thymosin. In some embodiments, the dose of alpha thymosin is at least about 0.5 mg (e.g., 1.6 mg), or at least about 3 mg (e.g., 3.2 mg), or at least about 5 mg (e.g., 6.4 mg). In certain embodiments, the thymosin peptide (e.g., TA1) is administered at a dose within the range of about 2 to about 8 mg. The thymosin peptide is generally administered from 1 to 4 times, such as once or twice. Where a plurality of alpha thymosin administrations are provided, the administrations may be spaced over a course of, for example, one week, ten days, two weeks, or one month. In some embodiments, at least two consecutive alpha thymosin administrations are spaced apart by a period of time ranging from about 5 days to about 10 days, e.g., about 7 days apart for approximately weekly administrations of TA1.

In certain embodiments, the thymosin peptide regimen may be administered from 1 to 10 days prior (e.g., from 5 to 8 days prior) to admittance to the hospital or an invasive medical procedure, and/or introduction of an invasive medical device, and again on the day of such an event, and optionally after the event, to thereby prevent or reduce the severity of any resulting infection from the anticipated pathogen exposure. The thymosin peptide may be administered about 7 days prior to the time of increased pathogen exposure (e.g., admittance to hospital or invasive medical procedure), and again on the day of such anticipated exposure.

In still other aspects, the invention provides a method for reducing the rate or incidence of hospital-acquired infection, by providing the regimen of alpha thymosin as described herein to at-risk patients. In such embodiments, the regimen of alpha thymosin peptide is initiated for at-risk patients upon admittance to a hospital or healthcare facility, especially where the patient is scheduled for a stay in the facility of greater than 5 days, or greater than 1 week, or greater than 2 weeks, and/or scheduled for an invasive medical procedure or in need of an invasive medical device. In certain embodiments, a TA1 administration is given to such patients about every five to ten days, or approximately weekly.

Other objects and aspects of the invention will be apparent from the following detailed description.

### DESCRIPTION OF THE FIGURES

FIG. 1 shows the number of mice reaching the desired antibody titer against 3 strains of influenza, upon receiving thymosin peptide at the indicated dose and at varying times with respect to Fluvirin® administration.

FIG. 2 shows the number of mice reaching the desired antibody titer upon receiving thymosin peptide at the indicated dose and at varying times with respect to vaccine administration (Fluvirin®). As shown, mice receiving thymosin peptide with the vaccine, and seven days prior to the vaccine, were all protected against three strains of influenza.

FIG. 3 shows the antibody titers achieved in ferrets with the human equivalent of 3.2 and 6.4 mg thymosin, when administered on the same day as an unadjuvanted vaccine, and in some cases seven days prior. An adjuvanted vaccine is shown as a positive control.

FIG. 4 shows results in patients with end-stage renal disease requiring hemodialysis. Patients received thymosin peptide on the day of vaccination (with Focetria™) and seven days prior. The left panel shows the percent of patients achieving seroprotection at day 21. The right panel shows the percent of patients achieving at least a four-fold increase in antibody titer at day 21.

FIG. 5 shows the results in patients with end-stage renal disease requiring hemodialysis. Patients received thymosin peptide on the day of vaccination (with Focetria™) and seven days prior. The graph shows the development of antibody titers over the 21 day period following vaccination

FIG. 6 shows percent seroconversion and antibody titer (geometric mean ratio, or GMR) in patients receiving influenza vaccine alone, or with regimens of 3.2 or 6.4 mg of TA1. Seroconversion is defined as negative pre-vaccination serum (i.e., HI titer <1:10) and post vaccination HI titer≧1:40 or a 4-fold increase from non-negative (≧1:10) pre-vaccination HI titer. GMR=ratios of day x/day 0 geometric mean HI titer. FIG. 6A shows results on Day 21. FIG. 6B shows results on day 42.

FIG. 7 shows percent seroconversion and geometric mean ratio (HI test) in patients receiving one dose of influenza vaccine, either alone or with regimens of 3.2 or 6.4 mg of TA1. FIG. 7A shows results on Day 21. FIG. 7B shows results on day 42.

FIG. 8 shows percent seroconversion and percent post-vaccination titer >1:40 in patients that were negative at baseline (HI titer <1:10). FIG. 8A shows results on Day 21. FIG. 8B shows results on day 42.

FIG. 9 shows seroconversion (HI test), with 95% confidence interval, in all patients over an 84 day period after influenza vaccination. For subjects receiving a second vaccination, the Day 21 titer was carried forward to Day 42 and 84.

FIG. 10 shows seroprotection (HI test), with 95% confidence interval, in all patients over an 84 day period after influenza vaccination. For subjects receiving a second vaccination, the Day 21 titer was carried forward to Day 42 and 84.

FIG. 11 shows Geometric Mean Titer (HI test), including 95% confidence interval, for all patients over an 84 day period after influenza vaccination. For subjects receiving a second vaccination, the Day 21 titer was carried forward to Day 42 and 84.

FIG. 12 shows Geometric Mean Ratio (HI test), including 95% confidence interval, for all patients over an 84 day period after influenza vaccination. For subjects receiving a second vaccination, the Day 21 titer was carried forward to Day 42 and 84.

### DETAILED DESCRIPTION OF THE INVENTION

The present invention provides methods for protecting, treating, or reducing the severity of infection, including hospital-acquired infection and infection related to invasive medical procedures or introduction of invasive medical devices. In certain embodiments, the invention involves treating or protecting from infection the immunodeficient or immunecompromised patient. As disclosed herein in the context of influenza vaccination, an efficient regimen of TA1 can prime the immune system for a greater or more rapid response to initial antigen or pathogen exposure, and provides such benefits for up to 50 days after exposure, which is sufficient to cover the time for most hospital stays, course of antibiotic therapy, and/or cycle of immunosuppressing drug.

The invention generally involves administering a regimen of alpha thymosin peptide to enhance immune responses to pathogen exposure, or potential pathogen exposure. Thymosin peptides include thymosin alpha 1 (“TA1”), and peptides having structural homology to TA1. TA1 is a peptide having the amino acid sequence (N-acetyl)-Ser-Asp-Ala-Ala-Val-Asp-Thr-Ser-Ser-Glu-11e-Thr-Thr-Lys-Asp-Leu-Lys-Glu-Lys-Lys-Glu-Val-Val-Glu-Glu-Ala-Glu-Asn-OH (SEQ ID NO: 1). The amino acid sequence of TA1 is disclosed in U.S. Pat. No. 4,079,137, the disclosure of which is hereby incorporated by reference. TA1 is a non-glycosylated 28-amino acid peptide having an acetylated N-terminus, and a molecular weight of about 3108. A synthetic version of TA1 is commercially available in certain countries under the trade name ZADAXIN.

TA1 circulates in serum at about 0.1 to 1.0 ng/ml. Peak plasma levels after injection of 3.2 mg of TA1 (about 40 μg/kg) is approximately 100 ng/ml, The half-life of TA1 in the circulation is about 2 hours.

Thymosin alpha was originally isolated from bovine thymus, where it was shown to reconstitute “immune function” in thymectomized animal models. Thymosin is thought to play a role in inflammatory and innate immune responses, and to facilitate discrimination of self from non-self in mammals. Activation of PAMP (pathogen-associated molecular patterns) ligands by thymosin leads to stimulation of intracellular signal transduction pathways resulting in expression of co-stimulatory molecules, pro-inflammatory cytokines, nitric oxide, and eicosanoids. Thymosin may fed, for example, dendritic cells, T cells, B cells, and NK cells.

Without intending to be bound by theory, it is believed that thymosin peptides (e.g., TA1), among other things, activate Toll-like Receptor 9 (TLR), resulting in increases in Th1 cells, B cells, and NK cells, thereby priming the immune system for an enhanced immune response. For example, TA1 may increase or enhance lymphocytic infiltration, secretion of chemotactic cytokines, maturation and differentiation of dendritic cells, secretion of thymopoeitic cytokines including IFN-α, IL-7, and IL-15, and B cell production of antibodies.

The thymosin peptides that find use with the invention include naturally occurring TA1 (e.g., TA1 purified or isolated from tissues), as well as synthetic TA1 and recombinant TA1. In some embodiments, the thymosin peptide comprises the amino acid sequence of SEC) ID NO:1 (where an acylated, e.g., acetylated. N-terminus is optional). In some embodiments, the thymosin peptide comprises an amino acid sequence that is substantially similar to TA1, and maintains the immunomodulatory activity of TA1. The substantially similar sequence may have, for example, from about 1 to about 10 amino acid deletions, insertions, and/or substitutions (collectively) with respect to TA1. For example, the thymosin peptide may have from about 1 to about 5 (e.g., 1, 2, or 3) amino acid insertions, deletions, and/or substitutions (collectively) with respect to TA1.

Thus, the thymosin peptide may comprise an abbreviated TA1 sequence, for example, having deletions of from 1 to about 10 amino acids, or from about 1 to 5 amino acids, or 1, 2 or 3 amino acids with respect to TA1. Such deletions may be at the N- or C-terminus, and/or internal, so long as the immunomodulatory activity of the peptide is substantially maintained. Alternatively, or in addition, the substantially similar sequence may have from about 1 to about 5 amino acid insertions (e.g., 1, 2, or 3 amino acid insertions) with respect to TA1, where the immunomodulatory activity of TA1 is substantially maintained. Alternatively, or in addition, the substantially similar sequence may have from 1 to about 10 amino acid substitutions, where the immunomodulatory activity is substantially maintained. For example, the substantially similar sequence may have from 1 to about 5, or 1, 2, or 3 amino acid substitutions, which may include conservative and non-conservative substitutions. In some embodiments, the substitutions are conservative. Generally, conservative substitutions include substitutions of a chemically similar amino acid (e.g., polar, non-polar, or charged). Substituted amino acids may be selected from the standard 20 amino acids or may be a non-standard amino acid (e.g., a conserved non-standard amino acid).

In some embodiments, the thymosin peptide comprises an amino acid sequence having at least 70% sequence identity to SEQ ID NO:1, while maintaining the immunomodulatory activity of TA1. For example, the thymosin peptide may comprise an amino acid sequence having at least 80%, 90%, or 95% sequence identity to SEQ ID NO:1. The thymosin peptide may comprise an amino acid sequence having 100% sequence identity to SEQ ID NO:1. In all cases, the N-terminus may be optionally acylated (e.g., acetylated) or alkylated, for example, with a C1-10 or C1-C7 acyl or alkyl group.

In certain embodiments, the substantially similar and homologous peptides described above may function at a level of at least about 50%, 70%, 80%, 90%, or about 100% relative to TA1 (SEQ ID NO:1).

The thymosin peptide may be prepared synthetically, for example, by solid phase synthesis, or may be made recombinantly and purified by known techniques.

The thymosin peptide may be provided in lyophilized form, and reconstituted with sterile (e.g., aqueous) diluent prior to administration. Formulations of thymosin peptide may be administered by subcutaneous injection, or other effective route.

In certain embodiments, the thymosin peptide is pegylated to increase its half-life in circulation. Such strategies for increasing the half-life of therapeutic proteins are well known.

In accordance with the invention, the thymosin peptide (e.g., TA1) is administered to a subject or patient with a regimen sufficient to enhance the immune response to pathogen exposure for a period of time of at least one week, at least one month, or at least two months, so as to protect patients facing an anticipated period of increased pathogen exposure, risk of infection, or expected immunodeficiency. The alpha thymosin regimen in various embodiments is an “efficient” regimen. That is, the regimen achieves its goal with relatively few administrations of alpha thymosin and/or by timing the administration of alpha thymosin with events anticipated to result in pathogen exposure or opportunism. The “event” is not a vaccination, but an exposure or increased susceptibility to the potential infectious agent. The efficient regimen of alpha thymosin is relatively convenient and comfortable for the patient, as well as more affordable and effective.

In some embodiments, the efficient regimen employs a relatively high dose of alpha thymosin (e.g., at least 1.6 mg, 3.2 mg, or 6.4 mg), with only 1, 2, 3, or 4 doses being administered, and in most embodiments, 3 doses or less. The alpha thymosin administrations may be spaced apart by about 5 to 9 days, and may be given weekly in some embodiments, as is described in greater detail herein. During the course of the regimen, the patient in some embodiments does not receive a vaccination.

In other embodiments, the efficient regimen employs a relatively high dose of alpha thymosin, and times the initiation of the regimen at about 1 to 10 days (but preferably 5 to 9 days) prior to an event predicted to result in pathogen exposure or opportunism. Exemplary events are described herein. In some of these embodiments, the efficient regimen involves from 1 to 4 administrations of alpha thymosin, such as 3 or less. The alpha thymosin administrations may be spaced apart by about 5 to 9 days, and may be given weekly in some embodiments. During the course of the regimen, the patient need not receive a vaccination.

In still other embodiments, the efficient regimen involves from 1 to 4 administrations of alpha thymosin, such as 3 or less, and the regimen is timed to begin prior to an event anticipated to lead to pathogen exposure or opportunism. For example, the efficient regimen may be initiated from 2 to 10 days prior to the event, such as from 5 to 10 days prior, and a second dose may be administered on the day of the event. The alpha thymosin administrations may be spaced apart by about 5 to 9 days, and may be given weekly in some embodiments. During the course of the regimen, the patient need not receive a vaccination.

In still other embodiments, the efficient regimen involves a relatively high dose of alpha thymosin, provided approximately weekly (e.g., every 5 to 9 days), for 2, 3, 4 or more weeks. During the course of the regimen, the patient need not receive a vaccination.

In still other embodiments, the patient receives 2 doses of thymosin alpha (such as 2-8 mg per dose), and such doses are spaced by about 5 to 10 days, or approximately weekly. This regimen may be repeated approximately monthly, or every other month, and may be particularly beneficial for protecting chronically ill and immunodeficient patients from Infection. Various types of immunodeficiency for which these embodiments find use are described herein.

As disclosed herein, as few as one or two alpha thymosin administrations are sufficient to provide a more robust immune response to antigen/pathogen exposure for up to about 50 days, which is sufficient to cover the length of time of most hospital stays and recuperative periods, as well as standard courses of antibiotic treatment and/or cycle of immune suppressing drugs.

The invention is applicable to both human and veterinary health. Thus, the subject is generally a mammal, such as a human, livestock (e.g., cow, horse, pig, sheep, etc.), or domestic mammal (e.g., cat or dog).

In certain embodiments, the subject is immunodeficient. An immunodeficient subject (e.g., a human subject) exhibits a reduced capacity to fight infectious disease and/or a reduced capacity to respond to pathogen exposure. Examples of such immunodeficient subjects include an elderly patient, newborn, leukemic or neutropenic patient, a patient on hemodialysis (e.g., for treatment of chronic renal disease), patient receiving immunosuppressant therapy, AIDS patient, diabetic patient, patient receiving chemotherapy or radiation therapy for cancer, immunodeficiency caused by a genetic defect, malnutrition, drug abuse, alcoholism, or other immunecompromising illness or condition.

In certain embodiments, the immunecompromised subject is elderly. As animals age, their immune response is reduced, and the robustness of the immune response is diminished due to the prevalence of low affinity antibody response. Accordingly, the subject in these embodiments may be a human patient over the age of 45, or over the age of 50. In some embodiments, the subject is a human patient 60 years of age or older, 65 years of age or older, or 70 years of age or older.

In some embodiments, the subject is at risk of a hospital-acquired infection. A hospital-acquired infection is an infection that develops while hospitalized. The medical term for a hospital-acquired infection is “nosocomial.” Since antibiotics are frequently used within hospitals, the microbes associated with nosocomial infections, and their resistance to antibiotics, can differ from isolates outside of the hospital. As used herein, a nosocomial infection is an infection that is not present or incubating in the host prior to admittance to the hospital, but generally manifests after about 2 days after admittance.

In one aspect of the invention, the regimen of thymosin peptide is administered to prevent infection, or reduce the severity of an infection, in a patient at risk for an infection. According to this aspect, the alpha thymosin regimen is used to prime the patient's immune system to provide a more rapid response to a pathogen exposure, which in some embodiments may be anticipated for the patient based upon a scheduled event.

For example, the subject may be scheduled for an invasive surgical procedure, and in these embodiments, the alpha thymosin regimen reduces the risk and/or severity of post-surgical infection. Generally, invasive medical procedures carry a risk of infection, and exemplary procedures include joint replacement, organ or tissue transplantation or graft, introduction of a prosthesis, tissue removal including a tumor or cancerous tissue, tonsillectomy, appendectomy, splenectomy, thymectomy, kidney removal, amputation, removal of bone marrow, or other invasive medical procedure. In such embodiments, the TA1 regimen may reduce the risk of endocarditis, bacteremia, sepsis, pneumonia, or osteomyelitis, or local infection of tissue around an incision site.

In certain embodiments, the patient may require assistance from an invasive medical device, which causes exposure of the body to microbes, and introduces an opportunistic environment. Thus, the device may lead to increased exposure to potential opportunists and pathogens. Such devices include without limitation, a ventilator, a urinary catheter, an arterial catheter, a feeding tube, i.v., stent, kidney dialysis, or artificial organ. In these embodiments, the alpha thymosin regimen helps to prime the patient's immune system to prevent or reduce the severity of any resulting infection.

In certain embodiments, the patient is in need, or is under assistance of a pulmonary ventilator, and the TA1 regimen helps to prime the patient's immune system, and retain the immune system in a primed state, so as to reduce the risk or severity of ventilator-associated pneumonia. Ventilator-associated pneumonia (VAP) occurs in patients on mechanical ventilation through an endotracheal or tracheostomy tube, and results from infection in the alveoli. Pseudomonas aeruginosa is the most common gram-negative bacterium causing VAP, and Pseudomonas has natural resistance to many antibiotics. Other causative species for VAP include Klebsiella pneumoniae, which has natural resistance to some beta-lactam antibiotics such as ampicillin and/or carbapenum, as well as cephalosporins and aztreonam. Serratia marcescens, Enterobacter sp., and Acinetobacter sp. may also be associated with VAP, and can also be resistant to antibiotics. In addition, there is an increasing association between Staphylococcus aureus (including MRSA) with VAP.

In certain embodiments, the subject is scheduled to undergo transplantation, followed by treatment with an immune suppressing drug, such as cyclosporine, tacrolimus, rapamycin, or agent that reduces production of antibodies. Thus, in certain embodiments the thymosin peptide regimen as described herein is initiated to boost the patient's development of antibodies prior to transplantation surgery and administration of immune suppressing drugs.

In some embodiments, the patient is on hemodialysis (e.g., due to chronic renal disease), or is scheduled to undergo hemodialysis. Since hemodialysis requires access to the circulatory system, patients undergoing hemodialysis may expose their circulatory system to microbes, which can lead to sepsis, an infection affecting the heart valves (endocarditis) or an infection affecting the bones (osteomyelitis). Thus, in certain embodiments the TA1 regimen as described herein is initiated to prepare a patient for hemodialysis.

In some embodiments, the patient is a cancer patient, and is undergoing or scheduled to initiate chemotherapy and/or radiation therapy, which often negatively affects the patient's immune system. Where the patient is undergoing or scheduled to initiate chemotherapy, the chemotherapy is generally one that has deleterious effects on the immune cells, and may include one or more alkylating agents (e.g., cisplatin, carboplatin, and ifosfamide), antimetabolite (5-fluorouracil or antifolate), topoisomerase inhibitor (e.g., camptothecin, etoposide), or taxane (e.g., paclitaxel), among others. in some embodiments, the alpha thymosin regimen is administered to prime the patient's immune system prior to cancer therapy.

In one exemplary embodiment, a regimen of alpha thymosin as described herein is provided to leukemic and/or neutropenic patients, thereby preventing or reducing the severity of catheter-related infection and/or bacteremia, such as are commonly caused by drug resistant Streptococcus aureus (e.g., MRSA and VRSA). In another exemplary embodiment, a regimen of alpha thymosin peptide as described herein is provided to bone marrow transplant patients, thereby preventing or reducing the severity of sepsis or pneumonia, such as those commonly caused by aspergillus, candida, or CMV. In still another embodiment, a regimen of alpha thymosin peptide as described herein is provided to organ (e.g., kidney) transplant recipients, to thereby prevent organ rejection, which is sometimes a result of CMV infection.

In another aspect, the invention provides a method for treating an infection. In this aspect, the patient is suspected of having an infection or has been diagnosed as having an infection, such as an acute respiratory, systemic, urinary, or local infection of the skin or a mucosal surface. The infection may be of bacterial, viral, fungal, or mixed or unknown etiology. The infection may be hospital-acquired, and may manifest as sepsis, pneumonia, urinary tract infection, endocarditis, osteomyelitis, or other condition.

In certain embodiments, the symptoms of infection are not present or are minor at the time of initiating the TA1 regimen, but the presence of the microorganism or illness is determined by culture, ELISA, or other diagnostic test. In such embodiments, the regimen of alpha thymosin helps to prime the immune system to more rapidly develop an antibody response capable of resolving the infection. In some embodiments, the alpha thymosin regimen is an efficient regimen that is provided concurrently with the standard antibacterial, antiviral, or antifungal therapy.

In certain embodiments, the patient shows signs and symptoms of infection. The infection, upon the appropriate diagnostic work, may be a respiratory infection such as respiratory syncytial virus (RSV), influenza virus, or bacterial pneumonia. In other embodiments, the infection is systemic, and may involve, for example, bacteremia, sepsis, or fungal infection, such as candidemia or aspergillis infection. In still other embodiments, the infection is a urinary tract infection, or a local infection of the skin or a mucosal surface, and may involve Staphylococcus aureus (e.g., a drug resistant S. aureus) or E. coli. The infection may result from severe injury, severe wound, or burn, and may be a post-surgical infection.

In certain embodiments, the patient (or a patient sample, susceptible site for infection, or immediate surrounding environment) has tested positive for the presence of a gram positive or gram negative bacteria, including one or more infectious organisms, including, but not limited to: Lysteria monocytoaenes, Pseudomonas sp. (e.g., P. aeruginosa), Serratia marcescens, Clostridium difficile, Staphylococcus aureus, Acinetobacter spp., Enterococcus sp., E. coil, Klebsiella sp., Streptococcus (e.g., S. pneumoniae), Haemophilus influenzae, and Neisseria meningitidis. In some embodiments, the infection involves, or an isolate is identified, as a drug resistant or multi-drug resistant microorganism, such as Staphylococcus aureus, Pseudomonas sp., Klebsiella sp., E. coli, and/or Clostridium Difficile. In certain embodiments, the infectious agent is a drug-resistant S. pneumoniae, including penicillin-resistant, methicillin-resistant, and/or quinolone-resistant (e.g., fluoroquinilone). In certain embodiments, the drug-resistant microorganism is methicillin-resistant or vancomycin-resistant Staphylococcus aureus (MRSA or VRSA), including intermediate resistant isolates, or is carbapenum-resistant E. coli, Klebsiella, or Pseudomonas including intermediate resistant isolates. The presence of such organisms may be determined or confirmed using diagnostics tests known in the art, or determined by a spike in the incidence of such infection at the healthcare facility.

In particular exemplary embodiments, the patient is a neutropenic patient inflicted with a Pseudomonas, Acinetobacter, or E. coli infection, and the infection may be drug resistant, or the patient is inflicted with ventilator-associated pneumonia, which may involve infection with Pseudomonas or Serratia, which may also show drug resistance.

The regimen of alpha thymosin may be administered concurrently with antibiotic therapy, including with beta-lactam antibiotic (e.g., methicillin, ampicillin, carbapenern, piperacillin); cephalosporin; fluoroquinolone (e.g., ciprofloxacin, levofloxacin, moxifloxacin), and/or macrolide (e.g., azithromycin, clarithromycin, dirithromycin, and erythromycin). The antibiotic therapy may be administered with additional therapeutics, such as a beta-lactamase inhibitor (tazobactam). In certain embodiments, alpha thymosin reduces the duration of the infection, and reduces the duration of required antibiotic treatment. In certain embodiments, the infection is determined to be resistant to such agent, prior to initiating alpha thymosin treatment. In certain embodiments, the alpha thymosin regimen is initiated, or continued, or repeated, after apparent resolution of the infection, to help prevent recurrence after antibiotic therapy is complete. An efficient regimen of alpha thymosin (e.g., 1, 2, or 3 doses) may span the full course of antibacterial therapy, and provide a boost in immune response for the entire period.

In certain embodiments, the patient has a viral infection selected from cytomegalovirus (CMV), RSV, influenza virus, herpes simplex virus type 1, and parainfluenza virus. The alpha thymosin regimen described herein may reduce the severity and/or duration of the viral infection or outbreak, and may be provided alongside the appropriate antiviral therapy, which may be a virus-neutralizing antibody or a small molecule inhibitor, such as Tamiflu. In certain embodiments, the alpha thymosin regimen is initiated, or continued, or repeated, after apparent resolution of the viral infection, to help prevent recurrence after other therapy is complete.

In still other embodiments, the patient has a fungal infection of Aspergillus (e.g., A. fumigatus) or Candida (e.g., Candida albicans), and these may also show resistance to antibiotic treatments. In certain embodiments, the thymosin peptide regimen is administered with antifungal treatment. Antifungal therapies include azole drug such as an imidazole (e.g., ketoconazole) or a triazole (e.g. fluconazole). In certain embodiments, the alpha thymosin regimen is initiated, or continued, or repeated, after apparent resolution of the infection, to help prevent recurrence after antifungal therapy is complete.

In certain aspects of the invention, the alpha thymosin regimen is part of an institutional program to reduce the rate or incidence of hospital-acquired infection, by initiating TA1 regimens for at-risk patients. At risk patients may include those described above for treatment and prevention of infection, and including immunecompromised patients and those scheduled for surgery or invasive medical devices. In such embodiments, the regimen may reduce the rate or incidence of bacterial, viral, or fungal infections, and which may manifest as a reduced incidence of sepsis, bacteremia, pneumonia (including VAP), RSV infection, endocarditis, osteomyelitis, transplant rejection due to infection, or post-surgical infection.

The regimen of alpha thymosin peptide involves administering the agent to the subject or patient at a dose sufficient to enhance antibody titers, and/or sufficient to speed the development of antibody titers, to pathogen exposure. For example, in various embodiments the thymosin peptide is administered to a human patient at a dose corresponding to at least about 0.5 mg (e.g., at least about 1.6 mg), at least about 3 mg (e.g., at least about 3.2 mg), or at least about 5 mg (e.g., at least about 6.4 mg) of TA1. The thymosin peptide may generally be administered within the range corresponding to about 0.1 to 20 mg of TA1, or about 1 to 10 mg of TA1, or about 2 to 10 mg of TA1, or about 2 to 8 mg of TA1, or about 2 to 7 mg of TA1. In certain embodiments, where an efficient regimen is desired, the dosage unit is within a range of 3 to 6.5 mg, such as about 3.2 or 6.4 mg of TA1. In certain embodiments, the TA1 dose is adjusted to the size of the patient, and may be provided at from 10 to 100 μg kg (e.g., about 20, 40, 60, or 80 μg/kg). Doses may be adjusted for the species of the subject or patient, but in each case, approximately correspond to the human equivalent of TA1 (mg/kg).

The thymosin peptide (e.g., TA1) may be administered by any effective route, including by subcutaneous injection, intramuscular injection, intravenous injection or infusion, and orally. In certain embodiments, the thymosin peptide is administered by subcutaneous injection or by intravenous infusion. Generally, the scheduled dose of thymosin may be administered as a single dose (e.g., injection), or may be spaced out over the course of 24 hours or less, for example, by continuous infusion or repeated injection of subdose, or the like. The scheduled dose of thymosin peptide may be administered as a single injection.

In some embodiments, such as for immobilized or hospitalized patients, the TA1 may be administered by continuous infusion. Continuous infusion of TA1 is described in detail in US 2005/0049191, the entire disclosure of which is hereby incorporated by reference. Briefly, continuous infusion of thymosin peptide maintains an immune stimulating-effective amount of a thymosin peptide in a patient's circulatory system for a longer period. The plasma half-life of subcutaneously injected TA1 is about two hours, and thus, according to certain embodiments, the thymosin peptide may be administered to the patient for treatment periods of at least about 6, 10, 12 hours, or longer, which may improve effectiveness in some embodiments. The infusion may be carried out by any suitable means, such as by minipump.

Alternatively, the thymosin peptide can be administered by a plurality of injections (sub-doses of thymosin peptide) on a treatment day, so as to substantially continuously maintain an immune stimulating-effective amount of the thymosin peptide in the patient's circulatory system for a longer period of time, Suitable injection regimens may include an injection every 2, 3, 4, 6, etc. hours on the day of administration (e.g., from 2 to 5 injections), so as to substantially continuously maintain the immune stimulating-effective amount of the thymosin peptide in the patient's circulatory system on the day of thymosin treatment.

The immune stimulating-effective amounts of a thymosin peptide (e.g. TA1) may be substantially continuously maintained in a patient's circulatory system by administering the TA1 peptide to the patient at a rate within a range of about 0.0001-0.1 mg/hr/kg patient body weight. Exemplary administration rates are within a range of about 0.0003-0.03 mg/hr/kg patient body weight. For continuous infusion, the TA1 peptide is present in a pharmaceutically acceptable liquid carrier, such as water for injection, or saline in physiological concentrations.

Whether for treating or preventing infection, the thymosin peptide regimen may be an efficient regimen, and involve administering alpha thymosin (e.g., TA1) from 1 to 4 times, or from 1 to 3 times, and in certain embodiments, the TA1 is administered only twice (e.g., on two treatment days). For example, the alpha thymosin peptide is administered prior to, along with and/or after an event predicted to result in pathogen exposure or introduction of an opportunistic environment, as described above. For example, the event may be admittance to a hospital or health care facility for a period of time (e.g., at least 3 days, at least one week, or at least ten days, or at least one month). In other embodiments, the event is a scheduled surgery or invasive medical procedure, as described. In other embodiments, the event is the placement of an invasive medical device as described. In still other embodiments, the event is kidney dialysis or initiation of chemotherapy or radiation therapy for cancer treatment (as described).

The timing of thymosin administration may be selected to enhance the immune response including antibody titers (e.g., the development or level of antibody titers) to cover a period of increased risk of infection. For example, in certain embodiments, the thymosin peptide administrations are given about 5 days to about 9 days apart, and in various embodiments are administered about 6, 7, or 8 days apart. The thymosin administrations may be given about 7 days apart (e.g., approximately weekly administration). in other embodiments, the thymosin peptide administrations are given 1, 2, 3, or 4 days apart.

In some embodiments, the alpha thymosin peptide is first administered prior to an event (as described), such as admittance to a healthcare facility, scheduled surgery, or placement of invasive medical device, and again on the day of the event, and optionally after the event. For example, thymosin peptide may be administered from 1 to 10 days prior to the event, such as from about 5 to about 9 days prior to the event, and again on the day of the event. The thymosin peptide may be administered about 7 days prior to the event, and again on the day of the event, and optionally within 2 to 10 days after the event (e.g., from 4 to 8 days after the event). For example, patients receiving two doses of TA1 in accordance with certain embodiments of the invention are likely to achieve a faster and/or larger response to pathogen exposure, and which may be protective for at least 21 days, at least 42 days, or longer.

In certain embodiments, such as where the patient, including an immunodeficient patient, shows signs or symptoms of a hospital-acquired infection or infection suspected of being drug resistant (including involving infectious agents and drug-resistant organisms described herein), the patient receives TA1 at a dose of from 2 to 8 mg (e.g., at 1.6, 3.2 or 6.4 mg per dose) either once or two times daily, or every other day, for from 3 to 14 days (e.g., 3, 5, 7, 10, or 14 days). Such regimen may be timed with respect to an event that places the patient at further risk for exacerbation of the infection or complicating illness, such as those events described herein (e.g., surgery, hemodialysis, initiation of cancer treatment, placement of medical device). For example, the event may be scheduled at a time between day 2 and day 10 of the regimen, including day 3, day 5, day 7, or day 10. The regimen may be concurrent with antibacterial, antiviral, or antifungal therapy, including with active agents described herein.

In one embodiment, the patient is hospitalized or admitted to a healthcare facility, and receives approximately weekly administration of TA1, at a dose between 2 and 8 mg (e.g., about 3.2 or 6.4 mg), to protect or reduce the severity of nosocomial illness or illness resulting from a medical procedure or medical device. The regimen may continue in some embodiments for two to four weeks. Where the patient is part of a healthcare facility's TA1 program, the invention results in a reduced incidence of nosocomial infection, reduced number of days in ICU and/or reduced antimicrobial therapy.

### EXAMPLES

### Example 1

### Enhancement of H1N1 Vaccination in Mice

### Summary

A study was conducted to determine the potential of TA1 (thymalfasin) to enhance the formation of anti-influenza antibodies in CD-1 mice following different vaccination schedules with the seasonal influenza vaccine Fluvirin® 2008-2009. The mice received either control article or vaccine on Study Days (SDs) 1 and 10 or SDs 8 and 17. The mice also received different doses of TA1 at different times in relation to the vaccine administration. Both the control article and vaccine were administered via intramuscular injection to both the right and left hind limbs; TA1 was administered by the intraperitoneal route. All mice were given a fixed dose of control/vaccine regardless of the body weight. The mice were observed twice daily for mortality, moribundity, general health, and signs of toxicity; body weights were recorded prior to dosing, Blood samples were collected on either SD 20 or 27 (ten days after final vaccine administration) and these samples were analyzed for HAI antibody production. Following the blood collection, all animals were euthanized and discarded without necropsy.

The results indicate that the HAI titer was greater in mice receiving both TA1 and FLUVIRIN vs. those receiving FLUVIRIN alone. In addition, the highest dose of TA1 used in this study (1.2 mg/kg) increased the titers more consistently when compared to the other doses. Furthermore, the best dosing schedule was administration of TA1 seven days prior to and on the day of FLUVIRIN vaccination on SD 8, as all animals achieved desired anti-influenza antibodies in all tester strains.

### Experimental Study

Thymosin alpha 1 (TA1; trade name ZADAXIN®) is approved and commercially available. TA1 is found naturally in the circulation and produced in the body's thymus gland. ZADAXIN® (a synthetic version of thymosin alpha 1) stimulates the immune system at least in part by affecting T cells and NK cells.

TA1 has an excellent safety record. In clinical studies to date, more than 3,000 patients, including adults, the elderly, and children, with viral hepatitis B and hepatitis C, primary immunodeficiency diseases, and numerous cancers have been treated with TA1 with virtually no drug-related side effects. Nor has there been any worsening of side effects when TA1 is combined with other agents such as interferon and chemotherapy. In animal studies, TA1 has been administered in doses as high as 800 times the recommended human dose with no evidence of adverse clinical signs.

Clinical trials have demonstrated that TA1 increases the response to influenza and hepatitis B vaccines in the elderly and hemodialysis patients; however, the treatment regimen has involved 8 injections of TA1 subsequent to vaccination. The current study was conducted to determine the potential of different doses and dosing regimens (primarily with fewer injections) of TA1 to enhance the formation of anti-influenza antibodies in CD-1 mice following two different vaccination schedules with the seasonal influenza vaccine Fluvirin® 2008-2009.

Appropriate numbers of male CD-1 mice were purchased from Charles River Laboratories. The animals weighed 25 to 40 grams and were 7 to 9 weeks of age at the first dose.

The control article was 0.9% Sodium Chloride for Injection, USP, and was stored at room temperature.

TA1 was diluted with phosphate buffered saline to the appropriate concentrations and stored at 2 to 8° C. until used.

Fluvirin® 2008-2009 was diluted with 0.9% Sodium Chloride for Injection, USP, to the appropriate concentration and used on day of formulation.

The study was divided into 2 cohorts, depending upon the vaccine dosing schedule; five mice/group were randomly assigned to each group. The first cohort of mice (20 groups) received control article or vaccine on Study Days (SD) 8 (Vaccine) and 17 (Boost) and the second cohort of mice (23 groups) received control article or vaccine on SDs 1 (Vaccine) and 10 (Boost). TA1 administration occurred as indicated in Tables 3 and 4.

The control article (0.9% Sodium Chloride for Injection, USP) and vaccine (9 μg/dose FLuvirin® 2008-2009) were both administered via intramuscular injection to both the right and left hind limbs at a fixed dose of 0.05 mL of control article/vaccine (regardless of the body weight).

TA1 (0.3, 0.6 or 1.2 mg/kg/dose) was administered by the intraperitoneal route at a dose volume of 1 mL/kg.

TABLE 1
Mouse/Ferret/Human Dosing Schedule

| | | |
|---|---|---|
| Human Dose | Mouse Dose | Ferret Dose |

| mg/person | mg/kg | mg/kg | mg/kg |
|---|---|---|---|
| 1.6 | 0.02 | 0.3 | 0.14 |
| 3.2 | 0.04 | 0.6 | 0.28 |
| 6.4 | 0.08 | 1.2 | 0.57 |

FDA-specified comparisons between equivalent dosing was used to determine mouse and ferret doses.

Animals were observed twice daily for mortality, moribundity, general health, and signs of toxicity. Animals were observed for skin and fur characteristics, injection sites, eye and mucous membranes, respiratory, circulatory, and autonomic and central nervous systems, somatomotor and behavior patterns. Body weights were recorded prior to dosing only.

Blood samples for analysis of influenza antibody titer (HAI analysis) were collected from all the animals via cardiac stick on SD 20 or SD 27 (ten days after final control article/vaccine administration). Following the blood collection, all animals were euthanized by CO₂ inhalation, exsanguinated and disposed of without necropsy.

HAI analysis was performed in triplicate against the 3 vaccine strains present in the Fluvirin® 2008-2009 vaccine (Florida [B], Brisbane 10 and Brisbane).

TABLE 2
Cohort 1 (Control Article/Vaccine Administered on SD 1 and 10)

| | | | TA 1 Dose Level |
|---|---|---|---|
| Group | Treatment | Time of TA 1 Administration | (mg/kg/dose) |
| 1 | Control Article | Not applicable - | 0 |
| | | Control article (saline) will be | |
| | | administered on SD 1 and 10 | |
| 2 | Vaccine only | Not applicable - | 0 |
| | | Vaccine will be administered on SD 1 and | |
| | | 10 | |
| 3 | Vaccine/ | TA 1 will be administered at the same | 0.3 |
| | TA 1 | time as the vaccine on SD 1 but will not | |
| | | be administered on SD 10 | |
| 4 | Vaccine/ | TA 1 will be administered at the same | |
| | TA 1 | time as the vaccine on SD 1 and 10 | |
| 5 | Vaccine/ | 1 hr before vaccine administration on SD | |
| | TA 1 | 1 and at the time of vaccine administration | |
| | | on SD 1 but not on SD 10 | |
| 6 | Vaccine/ | 1 hr before vaccine administration on SD | |
| | TA 1 | 1 and 10 and at the time of vaccine | |
| | | administration on SD 1 and SD 10 | |
| 7 | Vaccine/ | At the time of vaccine administration on | |
| | TA 1 | SD 1 and 1 hr after administration on SD | |
| | | 1 but not on SD 10 | |
| 8 | Vaccine/ | At the time of vaccine administration on | |
| | TA 1 | SD 1 and 10 and one hour after vaccine | |
| | | administration on SD 1 and 10 | |
| 9 | Vaccine/ | TA 1 will be administered at the same | 0.6 |
| | TA 1 | time as the vaccine on SD 1 but will not | |
| | | be administered on SD 10 | |
| 10 | Vaccine/ | TA 1 will be administered at the same | |
| | TA 1 | time as the vaccine on SD 1 and 10 | |
| 11 | Vaccine/ | 1 hr before vaccine administration on SD | 0.6 |
| | TA 1 | 1 and at the time of vaccine administration | |
| | | on SD 1 but not on SD 10 | |
| 12 | Vaccine/ | 1 hr before vaccine administration on SD | |
| | TA 1 | 1 and 10 and at the time of vaccine | |
| | | administration on SD 1 and SD 10 | |
| 13 | Vaccine/ | At the time of vaccine administration on | |
| | TA 1 | SD 1 and 1 hr after administration on SD 1 | |
| | | but not on SD 10 | |
| 14 | Vaccine/ | At the time of vaccine administration on | |
| | TA 1 | SD 1 and 10 and one hour after vaccine | |
| | | administration on SD 1 and SD 10 | |
| 15 | Vaccine/ | TA 1 will be administered at the same time | 1.2 |
| | TA 1 | as the vaccine on SD 1 but will not be | |
| | | administered on SD 10 | |
| 16 | Vaccine/ | TA 1 will be administered at the same time | |
| | TA 1 | as the vaccine on SD 1 and SD 10 | |
| 17 | Vaccine/ | 1 hr before vaccine administration on SD | |
| | TA 1 | 1 and at the time of vaccine administration | |
| | | on SD 1 but not on SD 10 | |
| 18 | Vaccine/ | 1 hr before vaccine administration on SD | |
| | TA 1 | 1 and 10 and at the time of vaccine | |
| | | administration on SD 1 and SD 10 | |
| 19 | Vaccine/ | At the time of vaccine administration on | |
| | TA 1 | SD 1 and 1 hr after administration on SD 1 | |
| | | but not on SD 10 | |
| 20 | Vaccine/ | At the time of vaccine administration on | |
| | TA 1 | SD 1 and 10 and one hour after vaccine | |
| | | administration on SD 1 and SD 10 | |

TABLE 3
Cohort 2 (Control Article/Vaccine Administered on SD 8 and 17)

| | | | TA 1 Dose Level |
|---|---|---|---|
| Group | Treatment | Time of TA 1 Administration | (mg/kg/dose) |
| 1 | Control Article | Not applicable - | 0 |
| | | Control article (saline) will be | |
| | | administered on SD 8 and 17 | |
| 2 | Vaccine only | Not applicable - | 0 |
| | | Vaccine will be administered on SD 8 and | |
| | | 17 | |
| 3 | Vaccine/ | TA 1 will be administered at the same | 0.3 |
| | TA 1 | time as the vaccine on SD 8 | |
| 4 | Vaccine/ | 1 hr before and at the same time as | |
| | TA 1 | vaccine administration on SD 8 | |
| 5 | Vaccine/ | 1 hr after and at the same time as vaccine | |
| | TA 1 | administration on SD 8 | |
| 6 | Vaccine/ | SD 7 - the day prior to and at the same | |
| | TA 1 | time as vaccine administration on SD 8 | |
| 7 | Vaccine/ | SD 9 - the day after and at the same time | |
| | TA 1 | as vaccine administration on SD 8 | |
| 8 | Vaccine/ | SD 1 - 7 days prior to and at the same | |
| | TA 1 | time as vaccine administration on SD 8 | |
| 9 | Vaccine/ | At the same time as vaccine | |
| | TA 1 | administration on SD 8 and 17 | |
| 10 | Vaccine/ | TA 1 will be administered at the same | 0.6 |
| | TA 1 | time as the vaccine on SD 8 | |
| 11 | Vaccine/ | 1 hr before and at the same time as | |
| | TA 1 | vaccine administration on SD 8 | |
| 12 | Vaccine/ | 1 hr after and at the same time as vaccine | |
| | TA 1 | administration on SD 8 | |
| 13 | Vaccine/ | SD 7 - the day prior to and at the | 0.6 |
| | TA 1 | same time as vaccine administration | |
| | | on SD 8 | |
| 14 | Vaccine/ | SD 9 - the day after and at the | |
| | TA 1 | same time as vaccine administration | |
| | | on SD 8 | |
| 15 | Vaccine/ | SD 1 - 7 days prior to and at the | |
| | TA 1 | same time as vaccine administration | |
| | | on SD 8 | |
| 16 | Vaccine/ | At the same time as vaccine | |
| | TA 1 | administration on SD 8 and 17 | |
| 17 | Vaccine/ | TA 1 will be administered at the | 1.2 |
| | TA 1 | same time as the vaccine on SD 8 | |
| 18 | Vaccine/ | 1 hr before and at the same time as | |
| | TA 1 | vaccine administration on SD 8 | |
| 19 | Vaccine/ | 1 hr after and at the same time as | |
| | TA 1 | vaccine administration on SD 8 | |
| 20 | Vaccine/ | SD 7 - the day prior to and at the | |
| | TA 1 | same time as vaccine administration | |
| | | on SD 8 | |
| 21 | Vaccine/ | SD 9 - the day after and at the | |
| | TA 1 | same time as vaccine administration | |
| | | on SD 8 | |
| 22 | Vaccine/ | SD 1 - 7 days prior to and at the | |
| | TA 1 | same time as vaccine administration | |
| | | on SD 8 | |
| 23 | Vaccine/ | At the same time as vaccine | |
| | TA 1 | administration on SD 8 and 17 | |

### Results

All animals survived until scheduled termination and there were no test article-related clinical/cageside observations or body weight effects noted in any animal.

When two doses of TA1 were administered to male CD-1 mice at different schedules in relationship to vaccination with Fluvirin® F02008-2009, the HAI titer was generally greater in animals receiving both TA1 and Fluvirin® 2008-2009 vs those receiving Fluvirin® 2008-2009 alone.

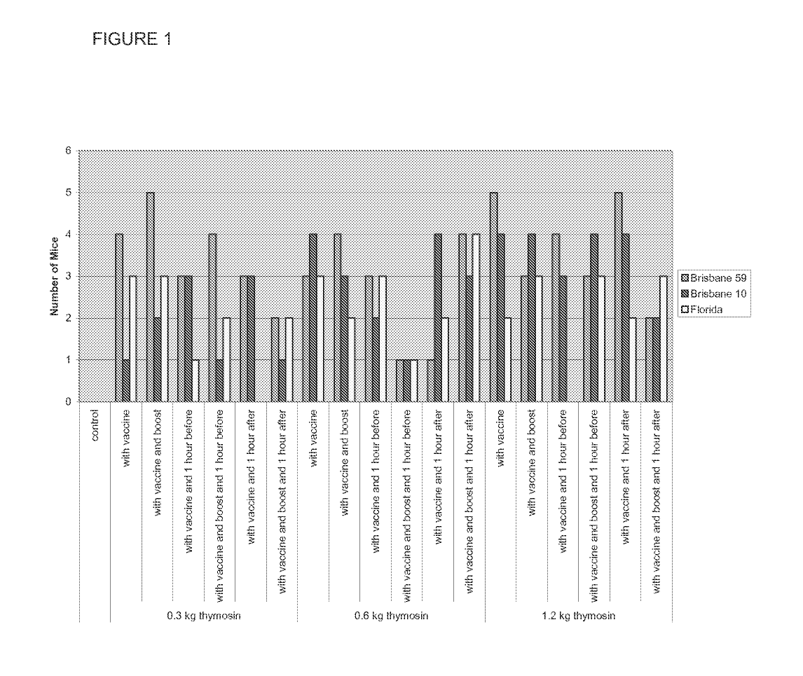

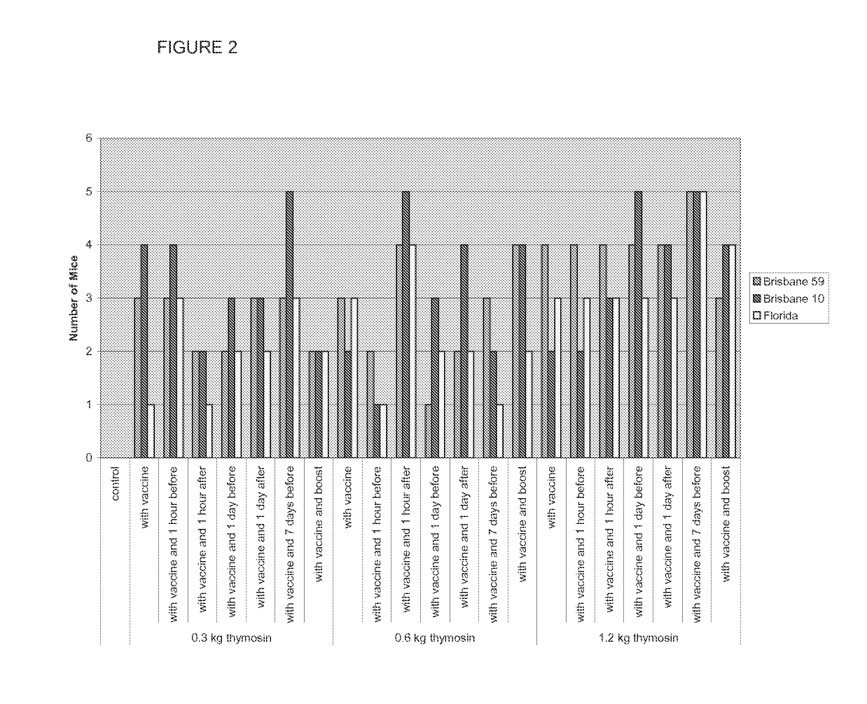

Under the different schedules investigated in the current study, the 1.2 mg/kg dose of TA1 increased the titers more consistently when compared to the other doses. See FIGS. 1 and 2. A dose of 1.2 mg/kg in mice is equivalent to a dose of approximately 6.4 mg in humans.

Furthermore, the best dosing schedule was TA1 administration seven days prior to and on day of Fluvirin® 2008-2009 vaccination on SD 8, as all animals achieved desired anti-influenza antibodies in all tester strains with this regimen. See FIGS. 1 and 2.

Thus, as determined by HAI titer assay, TA1 enhances the formation of anti-influenza antibodies in CD-1 mice vaccinated with two 9 μg doses of Fluvirin® 2008-2009. The most effective dosing regimen was 1.2 mg/kg TA1 given twice: seven days prior to and on the day of vaccination.

### Example 2

### Enhancement of H1N1 Vaccination in Ferrets

Thymosin has been shown to exert immunomodulation in several microbial and tumor settings by a variety of mechanisms which include potentiation of antibody responses. In the efforts to control the ongoing influenza pandemia caused by the new A/H1N1 virus of swine origin, a voluntary, mass vaccination will be implemented in most countries, and vaccines with or without adjuvants will be used. At least some of these vaccines will require a post-1 month booster dose to induce appreciable production of virus-neutralizing antibodies in most vaccines. Moreover, the availability of these vaccines for the whole target population is doubtful. It is therefore important to assess whether suitable doses of thymosin, administered separately but concomitantly with the influenza vaccine may potentiate the antibody responses to the virus.

### Experimental Study

Influenza-free ferrets are very responsive to influenza virus, and thus can be used to test protective anti-virus effects. In the experiments, potentiation of vaccine Immunogenicity was tested using both an adjuvanted influenza vaccine (Fluad: as a control) and non-adjuvanted influenza vaccine (Agrippal, labeled simply “vaccine” in the Table below).

5 groups of 4 ferrets received control article or vaccine on SD 0 (vaccine) and 21 (boost). TA1 administration occurred as indicated in Table 5. The proposed thymosin dosage was deduced with reference to published data in mice and humans, and taking into account the weight of the ferret. A pre-bleeding checked the negativity of anti-influenza titer.

The vaccine (either Agrippal influPozzi seasonal vaccine, non-adjuvanted, or Fluad, MF-59 adjuvanted) was administered via intramuscular injection to the right leg at a full human dose of 0.5 mL. TA1 (0.285 or 0.570 mg/kg/dose) was administered by the subcutaneous route at a dose volume that, using a scaling factor for ferret/human dosing, corresponding to approximate human doses of 3.2 or 6.4 mg/kg. Animals were observed twice daily for mortality, general health, and both local and systemic signs of toxicity and illness as well as behavior under the responsibility of a professional veterinarian. Body weights were recorded prior to dosing only.

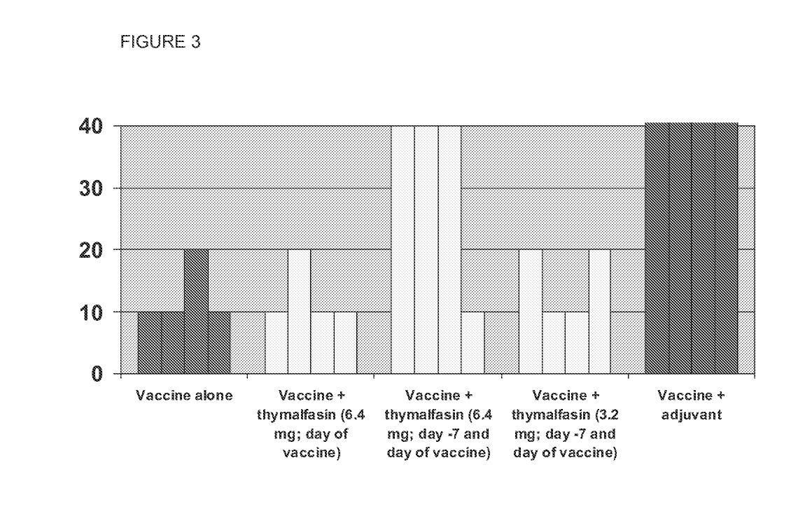

Blood samples for analysis of influenza antibody titer (hemagglutination-inhibition; HAI analysis) were collected from all the animals via a cardiac stick on SD 21 (prior to booster vaccine administration), SD 35, and SD 120. HAI analysis was performed in triplicate against the 3 vaccine strains (Florida [B], Brisbane 10 and Brisbane 59). Data for H1N1 A/Brisbane 59 are shown in FIG. 3. All ferrets had pre-existing antibodies against the H3N2 A/Brisbane 10.

TABLE 4
Study Design and Timeline

| Group | | | TA1 Dose |
|---|---|---|---|
| (n = 4) | Treatments | TA1 Administrations | (mg/kg) |
| 1 | Vaccine only | Not applicable - | — |
| | | vaccine administered on | |
| | | SD 0 and 21 | |
| 2 | Vaccine/TA1 | TA1 given 7 days before | 0.28 |
| | | and at the same time as | |
| | | vaccine on SD 0 | |
| 3 | Vaccine/TA1 | TA1 given 7 days before | 0.57 |
| | | and at the same time as | |
| | | vaccine on SD 0 | |
| 4 | Vaccine/TA1 | TA1 given at the same | 0.57 |
| | | time as vaccine on SD 0 | |
| | | and 21 | |
| 5 | Adjuvanted | Not applicable - | — |
| | vaccine only | vaccine administered on | |
| | | SD 0 and 21 | |

### Results

HAI titer (Day 21) in ferrets was generally greater in animals receiving two injections of TA1 plus vaccine versus those receiving vaccine alone (see FIG. 3). A 0.57 mg/kg dose of TA1 (equivalent to a human dose of approximately 6.4 mg/kg) administered seven days prior to and on the day of vaccination was the best performing dose/schedule, as ¾ animals received desired anti-influenza antibodies with this regimen. The titer persisted when evaluated 42 days after vaccination. Similarly, ferrets receiving TA1 on day 0 and +21 showed higher HAI titer after vaccine booster than those boosted without TA1. The antibody response in ferrets receiving adjuvanted vaccine grey exceeded that from non-adjuvanted vaccine, irrespective of TA1.

FIG. 3 shows the antibody titers in each group. A titer of 1:40 is considered protective. As shown, Thymalfasin at the human equivalent of 6.4 mg, given on day −7 and on the day of vaccination (without adjuvant), was protective. A 4-fold increase over vaccine alone was observed. Further, this dosing regimen produced protective titers in 3 of 4 animals.

TA1 appeared safe and well-tolerated, and no cage-side observations were noted. Thus, TA1 can enhance antibody response to non-adjuvanted influenza vaccine, a finding of relevance for vaccination of subjects with lowered response to vaccination, particularly the elderly

### Example 3

### Enhancer of H1N1 Vaccination in Hemodialysis Patients

The ability of thymosin TA1 to enhance immune response to the MF59 adjuvanted H1N1 influenza monovalent vaccine, Focetria™ was investigated. The study was conducted in hemodialysis patients. Patients with end-stage renal disease requiring hemodialysis, or other conditions that compromise the immune system, as well as the elderly, often do not develop sufficient antibodies to fight off infectious disease such as H1N1 influenza. Additionally, many patients that achieve protective titers initially are unable to sustain these for longer periods of time, making them susceptible to infection and requiring revaccination or booster shots.

The randomized, three-arm study was conducted in approximately 120 patients with end-stage renal disease who are on chronic dialysis. One cohort of patients received the H1N1 vaccine only, while the other two groups received either two low-dose injections of thymalfasin (TA1) (3.2 mg seven days prior to vaccination and on the day of vaccination), or two higher dose injections of thymalfasin (6.4 mg seven days prior to vaccination and on the day of vaccination). All patients who did not achieve an antibody titer of at least 1:40 on day 21 received a second H1N1 vaccination on that day. Dosing regimens are based on preclinical results obtained in the ferret and mouse models. Blood was drawn at days 0, 21, 42, 84, and 168. A second dose of the H1N1 vaccine was administered to any patient who did not reach the protective titer at 18-28 days from the first vaccination (8 subjects, or 25%, of the 32 subjects receiving vaccine alone; 2, or 7.1% of the 28 subjects receiving vaccine and 3.2 mg doses of TA1; and 2, or 6.3%, of the 32 subjects receiving vaccine and 6.4 mg doses of TA1).

The primary efficacy endpoint for the study is the proportion of patients who achieve seroconversion, specifically, a significant rise in specific antibody titers believed to be protective. In the context of this study using HI titers, “seroconversion” is defined as a change from negative pre-vaccination serum (e.g., HI titer <1:10) to post-vaccination titer≧1:40 or at least a four-fold increase in titers from baseline. Additionally, patients will be followed for six months to assess the durability of the protective titers. “Seroprotection” is defined as an HI titer of ≧1:40. The “Geometric Mean Ratio” (GMR) is the ratio of day x/day 1 geometric mean titers.

Thymalfasin treatment given with the H1N1 vaccine led to a highly statistical (p value ≦0.01) increase in the percentage of subjects who seroconverted at 21 days after vaccination, when compared to those who received the H1N1 vaccine alone. Specifically, at 21 days following vaccination, 89% of patients in the low-dose arm achieved seroconversion as did 88% of patients in the high-dose arm, compared to only 56% of patients in the vaccine-only arm.

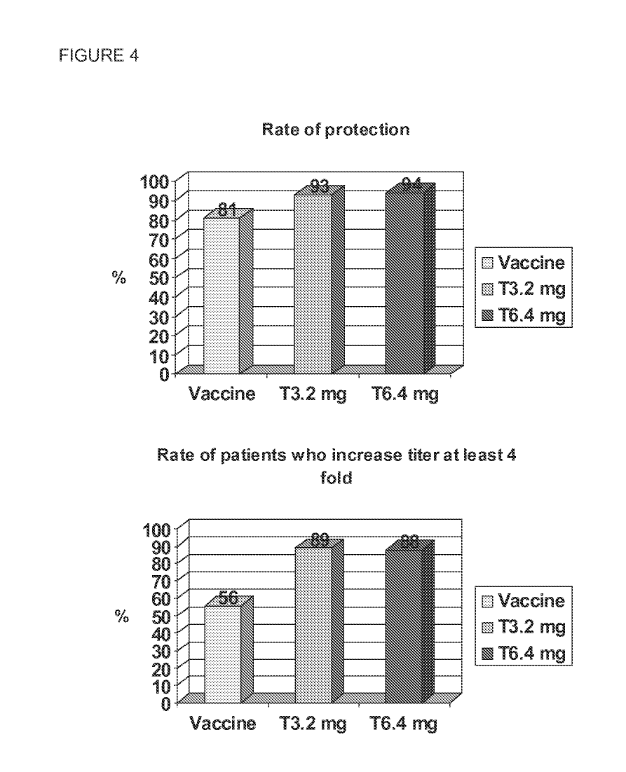

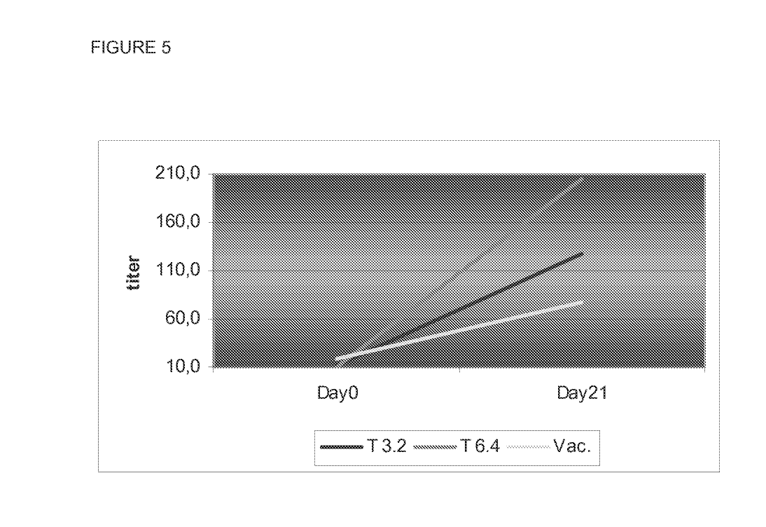

As illustrated in FIG. 5 (showing mean titer at baseline and at day 21), treatment with two doses of thymalfasin increases the mean titer in a dose-dependent fashion. FIG. 4 shows that the number of persons with seroprotection and the number of persons who seroconvert are greater with thymalfasin treatment.

Thymalfasin treatment given with the H1N1 vaccine led to a statistically significant (P value=0.04) increase in the percentage of subjects who seroconverted, also when evaluated at 42 days after vaccination, compared to those who received the H1N1 vaccine alone. In addition, the improvement in titers seen in thymalfasin-treated patients was maintained at this timepoint. Specifically, when measured 42 days following vaccination, 93% of patients in the low-dose arm and 94% of patients in the high-dose arm achieved seroconversion, compared to only 77% of patients in the vaccine only arm of the study. This increased seroconversion compares favorably with that seen at 21 days following vaccination.

The following tables summarizes microneutralization (MN) and seroconversion (SC) data through day 84 of the study.

TABLE 5
Overall Population:

| | | | |
|---|---|---|---|
| | V | V + T3.2 | V + T6.4 |
| CHMP criteria | N = 32 | N = 28 | N = 32 |

Day 21 MN test

| | | | |
|---|---|---|---|
| Percent with SC | 21.9 | 25 | 31.6 |
| Percent with MN ≧ 1:20 | 50 | 46.4 | 62.5 |
| GMR | 2.23 | 1.95 | 2.46 |

Day 42 MN test

| | | | |
|---|---|---|---|
| Percent with SC | 29 | 17.6 | 40.6 |
| Percent with MN ≧ 1:20 | 51.6 | 39.3 | 65.6 |
| | N = 31 | | |
| GMR | 2.27 | 1.72 | 2.33 |

Day 84 MN test

| | | | |
|---|---|---|---|
| Percent with SC | 22.6 | 17.6 | 40 |
| Percent with MN ≧ 1:20 | 41.9 | 35.7 | 66.7 |
| | N = 31 | | N = 30 |
| GMR | 2.15 | 1.62 | 2.32 |

Seroconversion is defined as negative pre-vaccination serum (i.e., MN titer < 1:10) and post-vaccination MN titer ≧ 1:20 or a 4-fold increase from non negative (≧1:10) pre vaccination MN titer. GMR = ratios of day x/day 0 geometric mean MN titer.

Only Subjects who received 1 vaccine dose

| | | | |
|---|---|---|---|
| | V | V + T3.2 | V + T6.4 |
| CHMP criteria | N = 26 | N = 26 | N = 30 |

Day 21 MN test

| | | | |
|---|---|---|---|
| Percent with SC | 26.9 | 26.9 | 36.7 |
| Percent with MN ≧ 1:20 | 57.7 | 50 | 63.3 |
| GMR | 2.61 | 2.1 | 2.61 |

Day 42 MN test

| | | | |
|---|---|---|---|
| Percent with SC | 32 | 15.4 | 43.3 |
| Percent with MN ≧ 1:20 | 56 | 42.3 | 66.7 |
| | N = 25 | | |
| GMR | 2.48 | 1.8 | 2.46 |

Day 84 MN test

| | | | |
|---|---|---|---|
| Percent with SC | 24 | 19.2 | 42.9 |
| Percent with MN ≧ 1:20 | 44 | 38.5 | 67.9 |
| | N = 25 | | N = 28 |
| GMR | 2.25 | 1.68 | 2.42 |

Only Subjects non-protected at the baseline:

| | | | |
|---|---|---|---|
| | V | V + T3.2 | V + T6.4 |
| CHMP criteria | N = 25 | N = 25 | N = 27 |

Day 21 MN test

| | | | |
|---|---|---|---|
| Percent with SC | 20 | 28 | 40.7 |
| Percent with MN ≧ 1:20 | 36 | 40 | 55.6 |
| GMR | 2.17 | 2.00 | 2.65 |

Day 42 MN test

| | | | |
|---|---|---|---|
| Percent with SC | 28 | 20 | 44.4 |
| Percent with MN ≧ 1:20 | 40 | 32 | 59.3 |
| GMR | 2.33 | 1.74 | 2.42 |

Day 84 MN test

| | | | |
|---|---|---|---|
| Percent with SC | 24 | 20 | 44 |
| Percent with MN ≧ 1:20 | 32 | 28 | 60 |
| | | | N = 25 |
| GMR | 2.36 | 1.62 | 2.40 |

Defined as negative pre-vaccination serum (i.e., MN titer < 1:10) or non negative (≧1:10) but non protected (i.e., MN titer ≦ 1:20)

Only Subjects negative at the baseline:

| | | | |
|---|---|---|---|
| | V | V + T3.2 | V + T6.4 |
| CHMP criteria | N = 19 | N = 18 | N = 19 |

Day 21 MN test

| | | | |
|---|---|---|---|
| Percent with MN ≧ 1:20 | 26.3 | 33.3 | 47.4 |
| GMR | 2.31 | 2.08 | 2.88 |

Day 42 MN test

| | | | |
|---|---|---|---|
| Percent with MN ≧ 1:20 | 36.8 | 22.2 | 52.6 |
| GMR | 2.73 | 1.68 | 2.54 |

Day 84 MN test

| | | | |
|---|---|---|---|
| Percent with MN ≧ 1:20 | 31.6 | 22.2 | 50 |
| | | | N = 18 |
| GMR | 2.88 | 1.71 | 2.42 |

Defined as negative pre-vaccination serum (i.e., MN titer < 1:10).

Only Subjects received 2 doses of vaccine:

| | | | |
|---|---|---|---|
| | V | V + T3.2 | V + T6.4 |
| CHMP criteria | N = 6 | N = 2 | N = 2 |

Day 21 MN test

| | | | |
|---|---|---|---|
| Percent with MN ≧ 1:20 | 16.7 | 0 | 50 |
| GMR | 1.12 | 1.00 | 1.00 |

Day 42 MN test

| | | | |
|---|---|---|---|
| Percent with MN ≧ 1:20 | 33.3 | 0 | 50 |
| GMR | 1.59 | 1.00 | 1.00 |

Day 84 MN test

| | | | |
|---|---|---|---|
| Percent with MN ≧ 1:20 | 33.3 | 0 | 50 |
| GMR | 1.78 | 1.00 | 1.19 |

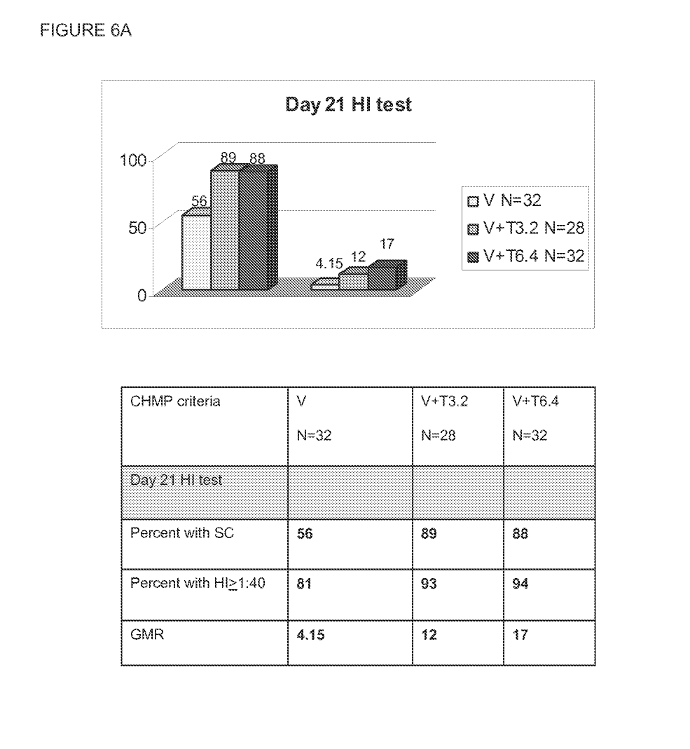

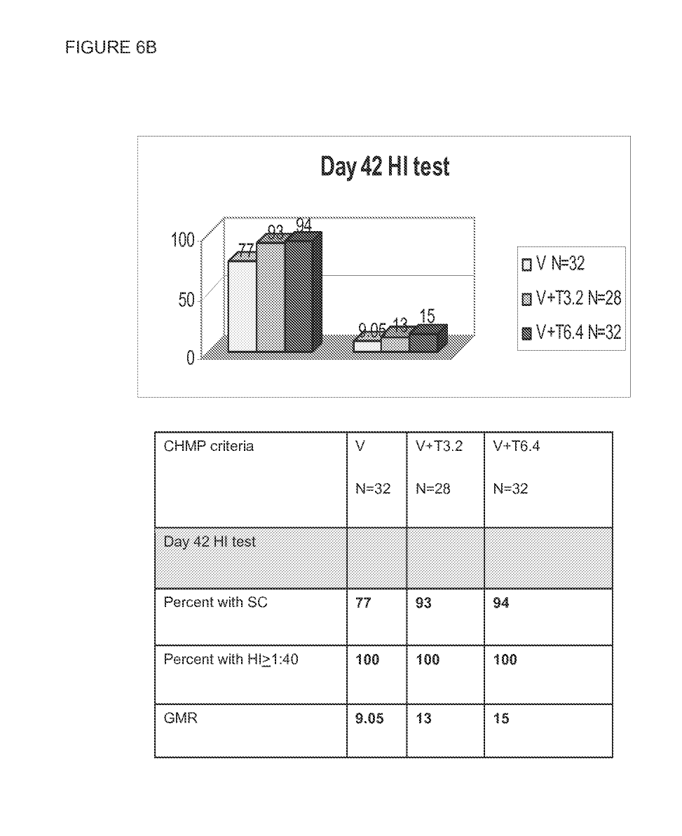

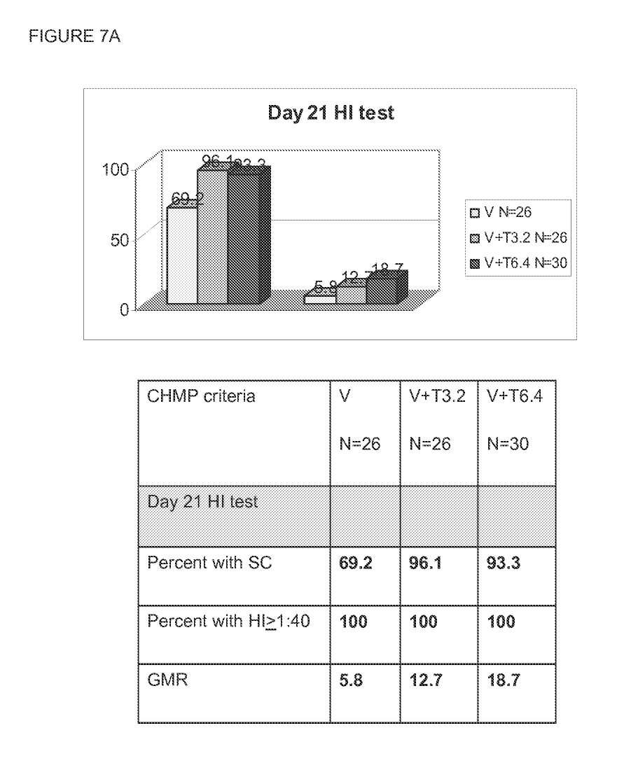

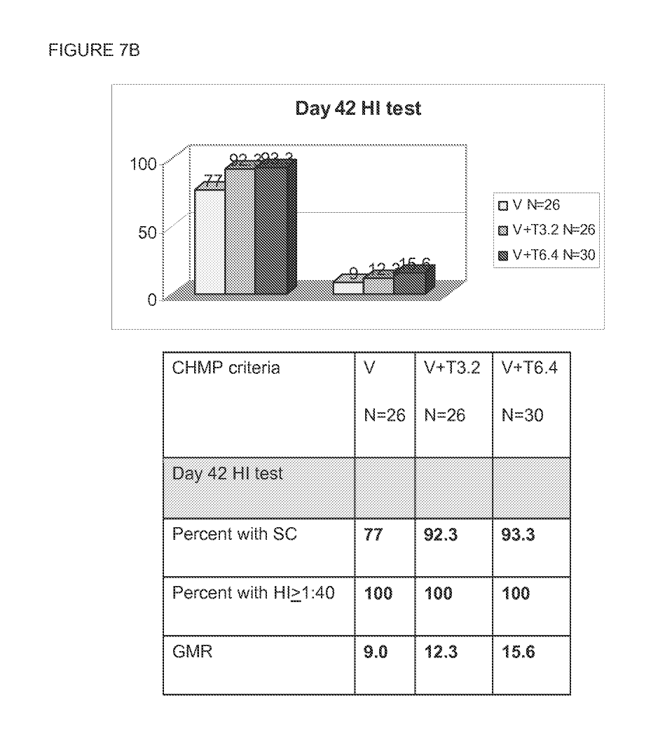

FIGS. 6 and 7 illustrate the results of HI test at days 21 and 42, and show a greater percent of patients with seroconversion and greater Geometric Mean Ratio with TA1 treatment.

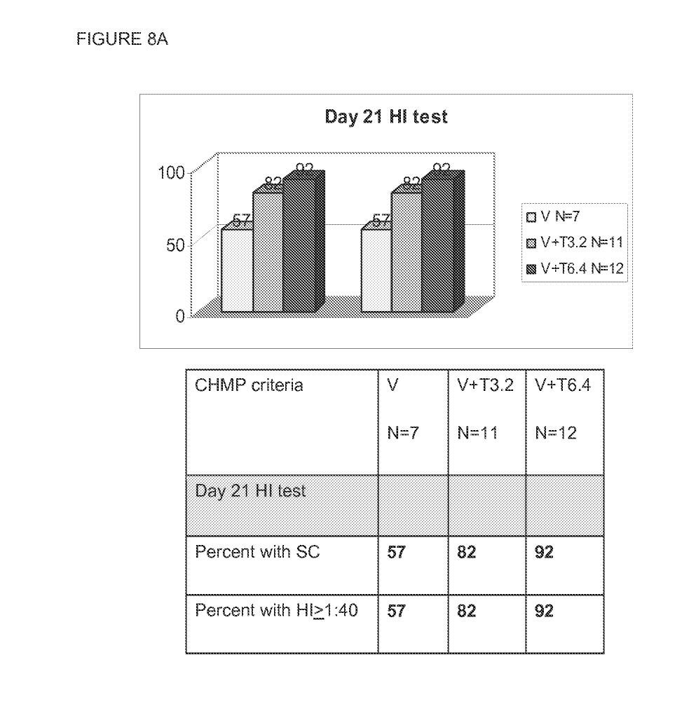

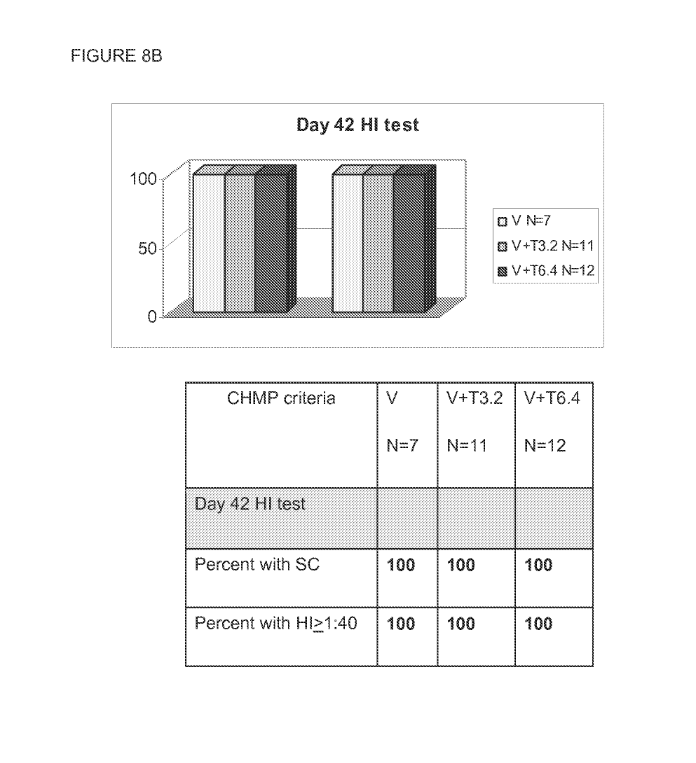

FIG. 8 illustrates the results on day 21 and 42, for patients that were negative at baseline. While all patients achieved seroconversion by day 42, at day 21, patients receiving TA1 were more likely to have achieved seroconversion.

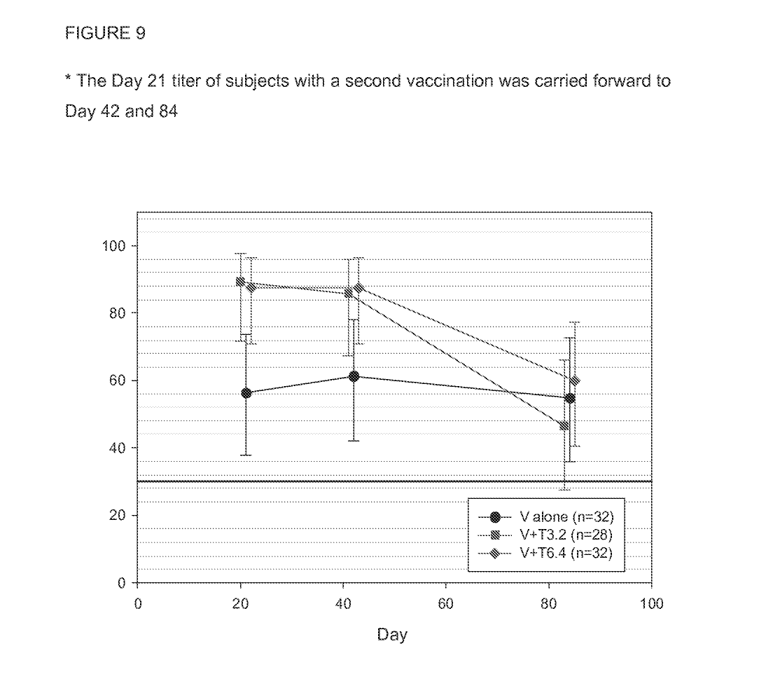

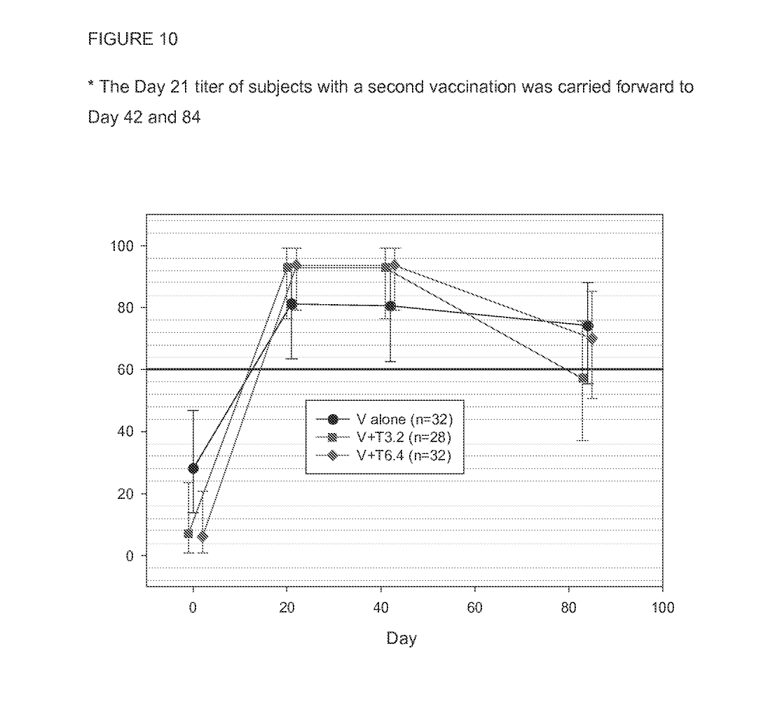

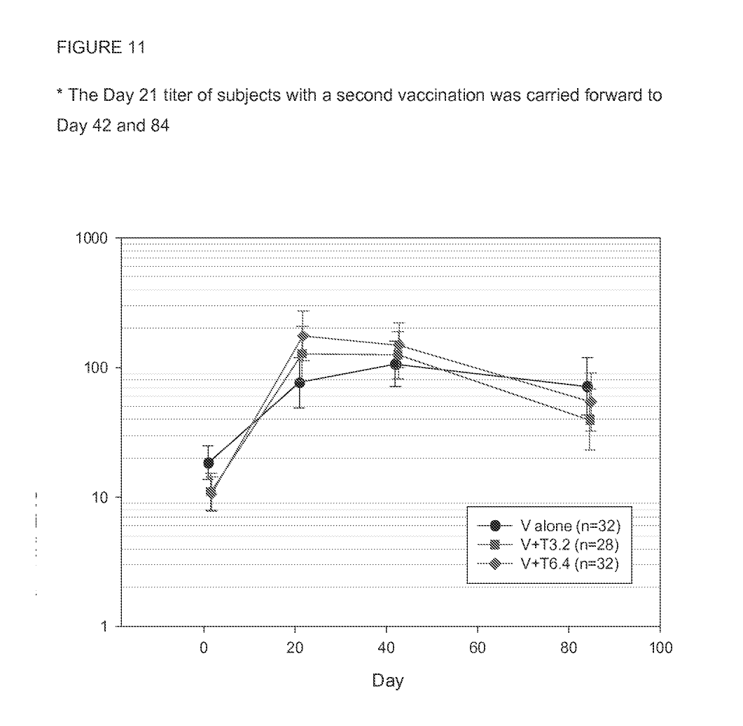

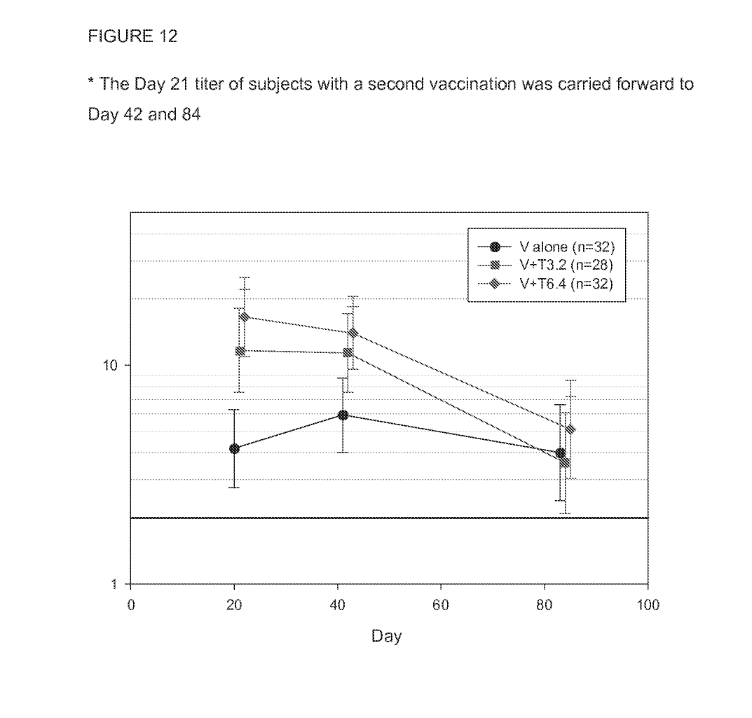

FIGS. 9 through 12 illustrate the results through day 84 of the study.

The study shows that two injections of TA1 given in addition to H1N1 adjuvanted vaccine led to an increase in vaccine efficacy, specifically: a more rapid response time, allowing patients to be protected sooner; as well as a better response than a single dose of vaccine alone or two vaccine injections.

### Example 4

### Protection from Infection

From the above results it was determined that efficient and cost effective infection treatment and prevention protocols could be developed with TA1, for example, by timing TA1 administrations for approximately weekly dosing, or with respect to anticipated antigen/pathogen exposures. Such exposures include admittance to a hospital, scheduled surgery or invasive medical procedure, placement of invasive medical device, and initiation of chemotherapy or radiation therapy.

1. A method for protecting a patient from infection, or reducing the severity of an infection, comprising, initiating an efficient regimen of alpha thymosin peptide prior to an event predicted to result in microbial exposure or opportunism, so as to prevent an infection or reduce the severity of a resulting infection.

2. The method of claim 1, wherein the patient is a human.

3. The method of claim 1, wherein the patient is immunodeficient.

4. (canceled)

5. The method of claim 1, wherein the patient is hospitalized for a period of from 3 days to about one month.

6-10. (canceled)

11. The method of claim 1, wherein the event is initiation of chemotherapy and/or radiation therapy for cancer, or admittance to a healthcare facility.

12. (canceled)

13. The method of claim 1, wherein the thymosin peptide is administered at a dose of at least about 0.5 mg.

14-18. (canceled)

19. The method of claim 1, wherein the regimen involves administering alpha thymosin from 1 to 4 times.

20-21. (canceled)

22. The method of claim 19, wherein at least two alpha thymosin peptide administrations are given about 5 days to about 9 days apart.

23. (canceled)

24. A method for treating an infection, comprising, administering an efficient regimen of alpha thymosin peptide so as to treat or reduce the severity of the infection.

25-26. (canceled)

27. The method of claim 24, wherein the infection is an acute respiratory infection, systemic infection, urinary tract infection, or local infection of the skin or a mucosal surface.

28-29. (canceled)

30. The method of claim 23, wherein the regimen of alpha thymosin is administered concurrently with antibacterial, antiviral, or antifungal therapy.

31. The method of claim 24, wherein the thymosin peptide is administered at a dose of at least about 0.5 mg.

32-36. (canceled)

37. The method of claim 24, wherein the regimen involves administering alpha thymosin peptide from 1 to 4 times.

38. (canceled)

39. The method of claim 37, wherein at least two thymosin peptide administrations are given about 5 days to about 9 days apart.

40. (canceled)

41. A method for reducing the rate or incidence of hospital-acquired infection, comprising initiating an alpha thymosin regimen for at-risk patients upon admittance to the hospital, the regimen comprising administration of alpha thymosin peptide at a frequency of once per every 5 to 10 days of hospitalization.

42. The method of claim 41, wherein the alpha thymosin peptide is administered approximately weekly.

43. The method of claim 41, wherein the at-risk patients are immunecompromised.

44-45. (canceled)

46. A method for treating a hospital-acquired infection or infection suspected of being drug resistant, comprising, administering alpha thymosin peptide at a dose of from 2 to 8 mg either once or two times daily, or every other day, for from 3 to 14 days.

47. The method of claim 46, wherein the patient is immune deficient.

48. The method of claim 46, wherein the infection involves an infectious microorganism selected from Lysteria monocytogenes, Pseudomonas sp., Serratia marcescens, Clostridium difficile, Staphylococcus aureus, Acinetobacter sp., E. coli, Klebsiella sp., Streptococcus, Haemophilus influenzae, and Neisseria meningitidis.

49-50. (canceled)
